(19) United States
(12) Patent Application Publication
(10) Pub. No.: US 2022/0096549 A1
(43) Pub. Date: Mar. 31, 2022

(54) CHIMERIC CYTOKINE RECEPTORS

(71) Applicant: Seattle Children's Hospital (dba Seattle Children's Research Institute), Seattle, WA (US)

(72) Inventors: Michael C. Jensen, Seattle, WA (US); Christopher P. Saxby, Seattle, WA (US)

(21) Appl. No.: 17/426,041

(22) PCT Filed: Feb. 6, 2020
(86) PCT No.: PCT/US2020/017067

§ 371 Date: Jul. 27, 2021

Related U.S. Application Data
(60) Provisional application No. 62/802,847, filed on Feb. 8, 2019.

(51) Int. Cl. A61K 35/17; C07K 14/725; C07K 14/715; C07K 14/54; C12N 9/12; A61P 35/00

(52) U.S. Cl. CPC A61K 35/17; C07K 14/7051; C07K 14/7155; C12Y 207/01021; C12N 9/1211; A61P 35/00; C07K 14/5418

(57) ABSTRACT

Some embodiments of the methods and compositions provided herein relate to chimeric cytokine receptors. In some embodiments, a chimeric cytokine receptor can include an IL-7 tethered to an extracellular IL-7 receptor domain, and an intracellular IL-21 receptor domain linked to the extracellular IL-7 receptor domain. In some embodiments, a T cell containing a chimeric cytokine receptor can be readily activated and/or expanded in the absence of an exogenous cytokine.

### PRIORITY AND CROSS-REFERENCE TO RELATED APPLICATIONS

This application claims the benefit of U.S. Provisional Application 62/802,847 filed on Feb. 8, 2019, which is hereby expressly incorporated by reference in its entirety.

### SEQUENCE LISTING IN ELECTRONIC FORMAT

The present application is being filed along with an Electronic Sequence Listing as an ASCII text file via EFS-Web. The Electronic Sequence Listing is provided as a file entitled SCRI215WOSEQLIST.txt, created and last modified on Feb. 3, 2020, which is 5,841 bytes in size. The information in the Electronic Sequence Listing is incorporated herein by reference in its entirety.

### FIELD

Some embodiments of the methods and compositions provided herein relate to chimeric cytokine receptors. In some embodiments, a chimeric cytokine receptor can include an IL-7 tethered to an extracellular IL-7 receptor domain, and an intracellular IL-21 receptor domain linked to the extracellular IL-7 receptor domain. In some embodiments, a T cell containing a chimeric cytokine receptor can be readily activated and/or expanded in the absence of an exogenous cytokine.

### BACKGROUND

In cell-based adoptive immunotherapy, T cells isolated from a patient can be modified to express synthetic proteins that enable the cells to perform new therapeutic functions after they are subsequently transferred back into the patient. Examples of such synthetic proteins are chimeric antigen receptors (CARs) and engineered T cell Receptors (TCR). An example of a currently used CAR is a fusion of an extracellular recognition domain (e.g., an antigen-binding domain), a transmembrane domain, and one or more intracellular signaling domains. Upon antigen engagement, the intracellular signaling portion of the CAR can initiate an activation-related response in an immune cell, such as release of cytolytic molecules to induce tumor cell death.

In preclinical models, CAR T cell therapy can be improved by supplementing with gamma chain cytokines, soluble factors that promote T cell growth and survival. However, systemic administration of cytokines to patients is not a therapeutically viable solution, as clinical trials have shown that this approach leads to toxic side-effects.

### SUMMARY

Some embodiments of the methods and compositions provided herein include a polynucleotide encoding a chimeric cytokine receptor polypeptide, wherein the chimeric cytokine receptor polypeptide comprises: an IL-7 tethered to an extracellular IL-7 receptor domain; a transmembrane domain; and an intracellular IL-21 receptor domain, wherein the transmembrane domain links the extracellular IL-7 receptor domain to the intracellular IL-21 receptor domain.

Some embodiments include a first nucleic acid encoding the IL-7; a second nucleic acid encoding a tether; a third nucleic acid encoding the extracellular IL-7 receptor domain, wherein the IL-7 is linked to the extracellular IL-7 receptor domain via the tether; a fourth nucleic acid encoding the transmembrane domain; and a fifth nucleic acid encoding an intracellular IL-21 receptor domain.

In some embodiments, the tether has a length from 3 amino acids to 30 amino acids.

In some embodiments, the transmembrane domain comprises an IL-7 receptor transmembrane domain.

In some embodiments, the first nucleic acid comprises a nucleotide sequence having at least 95% identity to SEQ ID NO:02. In some embodiments, the first nucleic acid comprises a nucleotide sequence of SEQ ID NO:02.

In some embodiments, the second nucleic acid comprises a nucleotide sequence having at least 95% identity to SEQ ID NO:03. In some embodiments, the second nucleic acid comprises a nucleotide sequence of SEQ ID NO:03.

In some embodiments, the third nucleic acid comprises a nucleotide sequence having at least 95% identity to SEQ ID NO:04. In some embodiments, the third nucleic acid comprises a nucleotide sequence of SEQ ID NO:04.

In some embodiments, the third and fourth nucleic acids together comprise a nucleotide sequence having at least 95% identity to SEQ ID NO:04. In some embodiments, the third and fourth nucleic acids together comprise a nucleotide sequence of SEQ ID NO:04.

In some embodiments, the fifth nucleic acid comprises a nucleotide sequence having at least 95% identity to SEQ ID NO:05. In some embodiments, the fifth nucleic acid comprises a nucleotide sequence of SEQ ID NO:05.

Some embodiments also include an inducible promoter. Some embodiments also include an inducible cytotoxic gene. In some embodiments, the cytotoxic gene encodes a protein selected from a thymidine kinase, thymidine kinase fused to thymidylate kinase, oxidoreductase, deoxycytidine kinase, uracil phosphoribosyltransferase, cytosine deaminase, or cytosine deaminase fused to uracil phosphoribosyltransferase. In some embodiments, the cytotoxic gene comprises a thymidine kinase. In some embodiments, the thymidine kinase comprises SR39TK.

Some embodiments also include a ribosomal skip sequence. In some embodiments, the ribosomal skip sequence comprises a T2A skip sequence.

In some embodiments, the polynucleotide encodes a transduction marker. In some embodiments, the marker comprises a truncated CD19 (CD19t).

Some embodiments of the methods and compositions provided herein include a vector comprising any one of the foregoing polynucleotides. In some embodiments, the vector comprises a viral vector. In some embodiments, the vector is selected from a lentiviral vector, an adeno-associated viral vector, or an adenoviral vector. In some embodiments, the vector comprises a lentiviral vector.

Some embodiments of the methods and compositions provided herein include a polypeptide encoded by any one of the foregoing polynucleotides.

Some embodiments of the methods and compositions provided herein include a cell comprising any one of the foregoing polynucleotides, or a protein encoded by any one of the foregoing polynucleotides.

Some embodiments also include a polynucleotide encoding a chimeric antigen receptor (CAR), or a CAR protein.

In some embodiments, the cell is a T cell. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex vivo.

Some embodiments of the methods and compositions provided herein include a method of treating or ameliorating a cancer in a subject, comprising: administering any one of the foregoing cells to the subject in need thereof.

In some embodiments, the treatment or amelioration of the cancer lacks co-administration of a cytokine to the subject.

Some embodiments also include co-administering a cytokine to the subject, wherein the dose of the cytokine administered is reduced compared to the dose of the cytokine co-administered to a subject who has been administered a cell comprising a CAR, which lacks the polynucleotide of any one of claims 1-23, or a protein encoded by the polynucleotide.

In some embodiments, the cytokine is selected from IL-7, IL-15, or IL-21. In some embodiments, the cytokine comprises IL-21.

In some embodiments, the cell is autologous to the subject.

In some embodiments, the cancer comprises a solid tumor such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the cancer comprises a brain cancer.

In some embodiments, the subject is mammalian. In some embodiments, the subject is human.

Some embodiments of the methods and compositions provided herein include a method of preparing a population of cells comprising a chimeric antigen receptor (CAR), comprising: (a) transducing a T cell with a polypeptide encoding a chimeric receptor, wherein the polypeptide comprises any one of the foregoing polynucleotides; (b) transducing the T cell with a polynucleotide encoding a CAR; and (c) culturing the transduced T cell under conditions sufficient to stimulate activation and expansion of the T cell, wherein the culture media comprises a reduced amount of an exogenous cytokine as compared to an amount sufficient to stimulate activation and expansion of the T cell lacking the polynucleotide encoding a chimeric receptor.

Some embodiments of the methods and compositions provided herein include a method of preparing a population of cells comprising a chimeric antigen receptor (CAR), comprising: (a) transducing a T cell with any one of the foregoing polynucleotides; (b) transducing the T cell with a polynucleotide encoding a CAR; and (c) culturing the transduced T cell under conditions to stimulate activation and expansion of the T cell, wherein the culture media lacks an exogenous cytokine.

In some embodiments, step (b) is performed before step (a).

In some embodiments, step (a) and (b) are performed concurrently.

In some embodiments, the cytokine is selected from IL-7, IL-15, or IL-21. In some embodiments, the cytokine comprises IL-21.

In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex vivo.

### BRIEF DESCRIPTION OF THE DRAWINGS

FIG. 1 depicts a schematic of a chimeric cytokine receptor for IL-21 signaling (CCRIL21) in a T cell. FIG. 1 (left panel) depicts a protein design of the CCRIL21, and FIG. 1 (right panel) depicts an exemplary function of the CCRIL21.

FIG. 2A depicts a schematic of a lentiviral construct encoding a CCRIL21.

FIG. 2B depicts a FACS analysis for CCRIL21-expressing CD8+ T cells, which were purified after transduction using CD19-specific magnetic sorting.

FIG. 3 depicts an analysis of CD8+ T cells for the presence of phosphorylated STAT3 (pSTAT3) or phosphorylated STAT5 (pSTAT5).

FIG. 4A depicts a graph showing the percentage of cells progressing through the cell cycle with error bars illustrating the standard deviation, for T cells either contacted with exogenous IL-21, or containing the chimeric cytokine receptor, CCRIL21.

FIG. 4B depicts a graph showing the percentage of cells undergoing apoptosis when T cells were either contacted with exogenous IL-21, or contained the chimeric cytokine receptor, CCRIL21.

FIG. 5A depicts a graph showing the percentage of tumor cells lysed by T cells containing a 806CAR, or T cells containing a 806CAR and the chimeric cytokine receptor, CCRIL21.

FIG. 5B depicts a FACS analysis for the presence of LAMP-1, GZMB, or PRF.

FIG. 6 depicts a graph showing the percentage of on-target and off-target tumor cells by T cells containing a 806CAR, or a 806CAR and the chimeric cytokine receptor, CCRIL2.

FIG. 7 depicts an analysis for the presence of BATF or T-bet.

FIG. 8A depicts a graph of tumor burden (measured by luminescence) over time for mice administered T cells containing either a 806CAR or a 806CAR and CCRIL21.

FIG. 8B depicts a Kaplan Meier graph showing the percent survival over time for tumor-bearing mice administered T cells containing either a 806CAR, or a 806CAR and CCRIL21.

FIG. 9A shows a graph of data demonstrating that CCRIL21-expressing B7H3CAR T cells are primed for increased cytotoxicity against tumor cells (Example 8).

FIG. 9B shows bar graphs of data demonstrating that CCRIL21-expressing CAR T cells are primed for increased cytotoxicity due to increased expression of effector proteins (Example 9).

FIG. 10 shows bar graphs of data demonstrating that CCRIL21 regulates effector function through key transcription factors (Example 10).

### DETAILED DESCRIPTION

Successful adoptive T cell therapy requires a robust expansion and persistence of administered T cells, and the environmental signals received by the T cell contribute heavily to these behaviors. In preclinical models, chimeric antigen receptor (CAR) T cell therapy can be improved by supplementing the therapy with gamma chain cytokines, which are soluble factors that promote T cell growth and survival. However, systemic administration of cytokines to patients is not a therapeutically viable solution, as clinical trials have shown that such intervention leads to toxic side-effects (Jeught V. et al. (2014) Oncotarget 6:1359-81). In order to confer the benefits of cytokine supplementation to CAR T cell therapy without incurring systemic toxicity, a panel of chimeric cytokine receptors has been engineered that provides T cell-intrinsic constitutive interleukin signaling. Chimeric cytokine receptors recapitulated the signaling events of a specific gamma chain cytokine. Moreover, CAR T cells containing certain chimeric cytokine receptors had unexpectedly enhanced activities.

Chimeric cytokine receptor-expressing CAR T cells were subjected to serial tumor challenges in vitro and the capacity of T cell groups to survive, proliferate and eliminate tumor cells was examined. It was found that chimeric cytokine receptor-expressing CAR T cells exhibited increased proliferation and survival in the absence of exogenous cytokines. Furthermore, specific chimeric cytokine receptors conferred differential effects on T cell mitogenesis and cytotoxicity, and these effects were traced to cytokine-specific regulation of key transcription factors. Moreover, an orthotopic glioblastoma xenograft mouse model was used to show that CAR T cells expressing these chimeric cytokine receptors exhibited markedly improved potency in vivo.

Optimum T cell activation and expansion requires three signals—T cell receptor activation, co-stimulation and stimulatory cytokines. In CAR T cells, the CAR can provide the first two signals, but the third signal remains dependent on environmental/exogenous cytokines, which can be scarce in a tumor microenvironment. While cytokine supplementation can improve the efficacy of CAR T cell therapy, systemic cytokine administration has led to toxicities in clinical trials. Developing methods to selectively provide the third signal to CAR T cells without or reducing systemic toxicities is clinically valuable.

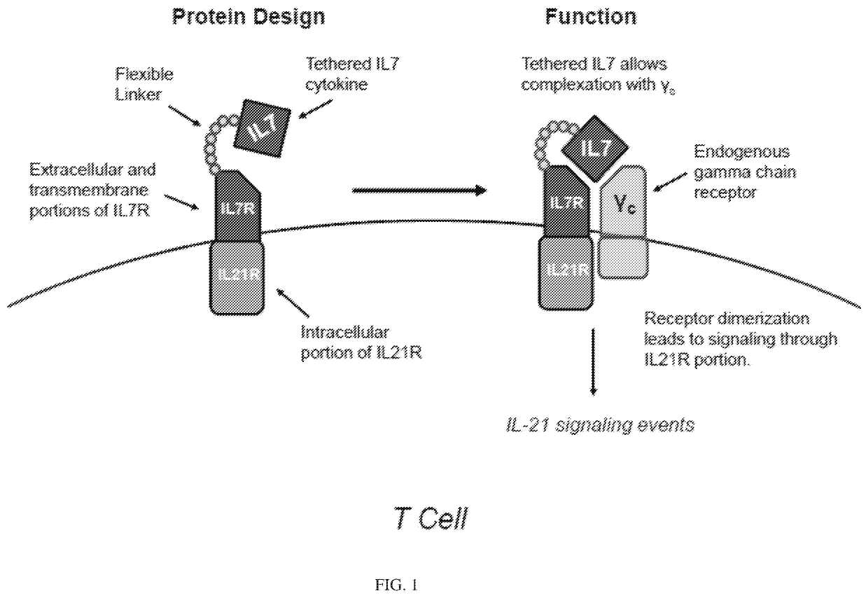

In some embodiments of the methods and compositions provided herein, a CAR T cell is engineered to include a chimeric receptor for interleukin 21 (CCRIL21). CCRIL21 provides stimulatory cytokine signals to the CAR T cell and improves the efficacy of CART cell therapy. FIG. 1 depicts a CCRIL21 in a T cell and shows a protein design (left panel), which includes a chimeric receptor protein comprising an extracellular domain and transmembrane domain of an IL-7 receptor and an intracellular domain of an IL-21 receptor, linked via a flexible linker to a tethered IL-7 cytokine. FIG. 1 (right panel) shows an exemplary function of the CCRIL21 in which the tethered IL-7 complexes with an endogenous gamma chain receptor, and receptor dimerization activates intracellular signaling via the IL-21 receptor domain.

In vitro primary human T cell data has demonstrated that CCRIL21 expressing CD8+ T cells: induced signaling downstream of IL-21 signaling, promoted cytokine independent proliferation and survival, and conferred increased cytotoxicity and effector functions. In vivo orthotopic glioblastoma data from mice has demonstrated that CCRIL21 CAR T cells significantly improved tumor clearance and promoted overall survival as compared to CAR T cells without CCRIL21.

### Terms

Terms in the disclosure herein should be given their plain and ordinary meaning when read in light of the specification. One of skill in the art would understand the terms as used in view of the whole specification.

As used herein, “a” or “an” may mean one or more than one.

As used herein, the term “about” indicates that a value includes the inherent variation of error for the method being employed to determine a value, or the variation that exists among experiments.

As used herein, “chimeric receptor” can include a synthetically designed receptor comprising a ligand binding domain of an antibody or other protein sequence that binds to a molecule associated with a disease or disorder and is linked, preferably via a spacer domain, to one or more intracellular signaling domains of a T cell or other receptors, such as a costimulatory domain. Chimeric receptors can also be referred to as artificial T cell receptors, chimeric T cell receptors, chimeric immunoreceptors, or chimeric antigen receptors (CARs).

As used herein, “chimeric cytokine receptor” can include a synthetically designed receptor comprising a cytokine tethered to an extracellular domain of a cytokine receptor polypeptide, a transmembrane domain, and an intracellular cytokine receptor domain linked to the extracellular domain of a cytokine receptor polypeptide via the transmembrane domain. In some embodiments, the cytokine can be selected from a type I cytokine receptor, such as IL-2, IL-4, IL-7, IL-9, IL-13, IL, 15, or IL-21. In some embodiments, the extracellular domain of a cytokine receptor polypeptide can be derived from a type I cytokine receptor, such as IL-2, IL-4, IL-7, IL-9, IL-13, IL, 15, or IL-21. In some embodiments, the transmembrane domain can be derived from a type I cytokine receptor, such as IL-2, IL-4, IL-7, IL-9, IL-13, IL, 15, or IL-21. In some embodiments, the intracellular domain of a cytokine receptor polypeptide can be derived from a type I cytokine receptor, such as IL-2, IL-4, IL-7, IL-9, IL-13, IL, 15, or IL-21.

As used herein, “chimeric antigen receptor” (CAR), also known as chimeric T-cell receptors, can refer to artificial T-cell receptors that are engineered receptors, which graft an arbitrary specificity onto an immune effector cell. These receptors can be used to graft the specificity of a monoclonal antibody onto a T-cell, for example; with transfer of their coding sequence facilitated by retroviral vectors, or any other suitable gene delivery system. The structure of the CAR can comprise single-chain variable fragments (scFv) derived from monoclonal antibodies, fused to CD3-zeta transmembrane and endodomain. Such molecules result in the transmission of a zeta signal in response to recognition by the scFv of its target. Some alternatives utilize a gene delivery vector having a self-inactivating transposase system. In some alternatives, the gene delivery vector further comprises a sequence for at least one protein. In some alternatives, the protein is a chimeric antigen receptor. Chimeric receptors can also be referred to as artificial T cell receptors, chimeric T cell receptors, chimeric immunoreceptors, and/or CARs. These CARs are engineered receptors that can graft an arbitrary specificity onto an immune receptor cell. CARs may include the antibody or antibody fragment, spacer, signaling domain, and/or transmembrane region. However, due to the surprising effects of modifying the different components or domains of the CAR, such as the epitope binding region (for example, antibody fragment, scFv, or portion thereof), spacer, transmembrane domain, and/or signaling domain), the components of the CAR are described herein in some contexts to include these features as independent elements. The variation of the different elements of the CAR can, for example, lead to stronger binding affinity for a specific epitope.

Artificial T-cell receptors, or CARs, can be used as a therapy for cancer or viral infection using a technique called adoptive cell transfer. T-cells are removed from a subject and modified so that they express receptors specific for a molecule displayed on a cancer cell or virus, or virus-infected cell. The genetically engineered T-cells, which can then recognize and kill the cancer cells or the virus infected cells or promote clearance of the virus, are reintroduced into the subject. In some alternatives, the gene delivery vector can comprise a sequence for a chimeric antigen receptor. In some alternatives, a method of generating engineered multiplexed T-cells for adoptive T-cell immunotherapy is provided. In the broadest sense the method can comprise providing the gene delivery vector of any one of the alternatives described herein, introducing the gene delivery vector into a T-cell, and selecting the cells comprising the gene delivery vector, wherein selecting comprises isolating the T-cells expressing a phenotype under selective pressure.

T-cell co-stimulation is desired for development of an effective immune response and this event occurs during the activation of lymphocytes. A co-stimulatory signal, is antigen non-specific and is provided by the interaction between co-stimulatory molecules expressed on the membrane of the antigen bearing cell and the T-cell. Co-stimulatory molecules can include but are not limited to CD28, CD80, or CD86. In some alternatives, a method for generating engineered multiplexed T-cells for adoptive T-cell immunotherapy is provided. In some alternatives, the T-cell is a chimeric antigen receptor bearing T-cell. In some alternatives, the chimeric antigen receptor bearing T-cell is engineered to express co-stimulatory ligands. In some alternatives, methods are provided for treating, inhibiting, or ameliorating cancer or a viral infection in a subject. In the broadest sense, the method can comprise administering to the subject a T-cell of any of the alternatives described herein. In some of these alternatives, the subject is an animal, such as domestic livestock or a companion animal and in other alternatives, the subject is a human. In some of these alternatives, the chimeric antigen bearing T-cell is engineered to express a co-stimulatory molecule. In some alternatives, the gene delivery vector comprises a sequence for at least one co-stimulatory molecule. In some alternatives, the gene delivery vector is at least 1 kB to 20 kB.

As described herein, “genetically modify” and “genetically modified” can include a process for modifying an organism or a cell such as a bacterium, T-cell, bacterial cell, eukaryotic cell, insect, plant or mammal with genetic material, such as nucleic acid, that has been altered using genetic engineering techniques. For example, a nucleic acid such as DNA can be inserted in the host genome by first isolating and copying the genetic material of interest using molecular cloning methods to generate a DNA sequence, or by synthesizing the DNA, and then inserting this construct into the host organism. Genes can also be removed, or “knocked out”, using a nuclease. Gene targeting is a different technique that uses homologous recombination to change an endogenous gene, and can be used to delete a gene, remove exons, add a gene, or introduce point mutations.

Genetic modification performed by transduction is described herein. “Transduction” refers to methods of transferring genetic material, such as, for example, DNA or RNA, to a cell by way of a vector. Common techniques use viral vectors, electroporation, and chemical reagents to increase cell permeability. The DNA can be transferred by a virus, or by a viral vector. Described herein are methods for modifying immune CD4+ and/or CD8+ T-cells. In order to achieve high expression of therapeutic genes and/or to increase the amount of chimeric antigen receptors on a cell surface, for example, T-cells are transduced with genetic material encoding a protein or a chimeric antigen receptor. T-cells can be genetically modified using a virus, for example. Viruses commonly used for gene therapy are adenovirus, adeno-associated virus (AAV), retroviruses or lentiviruses, for example.

Various transduction techniques have been developed, which utilize recombinant infectious virus particles for delivery of the nucleic acid encoding a chimeric antigen receptor. This represents a currently preferred approach to the transduction of T lymphocytes. As described herein, the viral vectors used for transduction can include virus vectors derived from simian virus 40, adenoviruses, AAV, lentiviral vectors, or retroviruses. Thus, gene transfer and expression methods are numerous but essentially function to introduce and express genetic material in mammalian cells. Several of the above techniques can be used to transduce hematopoietic or lymphoid cells, including calcium phosphate transfection, protoplast fusion, electroporation, or infection with recombinant adenovirus, adeno-associated virus, lentivirus, or retrovirus vectors. Primary T lymphocytes have been successfully transduced by electroporation and by retroviral or lentiviral infection. As such, retroviral and lentiviral vectors provide a highly efficient approach for gene transfer to eukaryotic cells, such as T-cells. Moreover, retroviral or lentiviral integration takes place in a controlled fashion and results in the stable integration of one or a few copies of the new genetic information per cell. As described herein, the cells can be transduced in situ.

As used herein, a “vector” or “construct” can include a nucleic acid used to introduce heterologous nucleic acids into a cell that can also have regulatory elements to provide expression of the heterologous nucleic acids in the cell. Vectors include but are not limited to plasmid, minicircles, yeast, or viral genomes. In some embodiments, the vectors are plasmid, minicircles, viral vectors, DNA or mRNA. In some embodiments, the vector is a lentiviral vector or a retroviral vector. In some embodiments, the vector is a lentiviral vector. As used herein, “Vpx” can include a virion associated protein that is encoded by HIV type 2 and in some simian immunodeficiency virus strains. Vpx can enhance HIV-2 replication in humans. Lentiviral vectors packaged with Vpx protein can led to an increase in the infection of myeloid cells, when used in transfections. In some embodiments, the lentiviral vector is packaged with a Vpx protein. As used herein, “Vpr” protein can refer to Viral Protein R, which is a 14 kDa protein, which plays an important role in regulating nuclear import of the HIV-1 pre-integration complex and is required for virus replication in non-dividing cells. Non-dividing cells can include macrophages, for example. In some embodiments, the lentiviral vector can be packaged with a Vpr protein, or a Vpr protein portion thereof. In some embodiments, the lentiviral vector is packaged with a viral accessory protein. In some embodiments, the viral accessory protein is selected from the group consisting of Vif, Vpx, Vpu, Nef and Vpr. These accessory proteins such as, for example vif, Vpx, vpu or nef interact with cellular ligands to act as an adapter molecule to redirect the normal function of host factors for virus-specific purposes. HIV accessory proteins are described in Strebel et al. (“HIV Accessory Proteins versus Host Restriction Factors, Curr Opin Virol. 2013 December; 3(6): 10.1016/j.coviro.2013.08.004; hereby expressly incorporated by reference in its entirety).

As used herein, a “promoter” can include a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. Without being limiting, these promoter elements can include RNA polymerase binding sites, TATA sequences, CAAT sequences, or differentiation-specific elements (DSEs; McGehee et al., Mol. Endocrinol. 7:551 (1993); hereby expressly incorporated by reference in its entirety), cyclic AMP response elements (CREs), serum response elements (SREs; Treisman et al., Seminars in Cancer Biol. 1:47 (1990); hereby expressly incorporated by reference in its entirety), glucocorticoid response elements (GREs), or binding sites for other transcription factors, such as CRE/ATF (O'Reilly et al., J. Biol. Chem. 267:19938 (1992); hereby expressly incorporated by reference in its entirety), AP2 (Ye et al., J. Biol. Chem. 269:25728 (1994); hereby expressly incorporated by reference in its entirety), SP1, cAMP response element binding protein (CREB; Loeken et al., Gene Expr. 3:253 (1993); hereby expressly incorporated by reference in its entirety) or octamer factors (see, in general, Watson et al., eds., Molecular Biology of the Gene, 4th ed. (The Benjamin/Cummings Publishing Company, Inc. 1987; hereby expressly incorporated by reference in its entirety)), and Lemaigre and Rousseau, Biochem. J. 303:1 (1994); hereby expressly incorporated by reference in its entirety). As used herein, a promoter can be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known.

As used herein, “treat,” “treating,” “treated,” or “treatment” can refer to both therapeutic treatment and prophylactic or preventative treatment depending on the context.

As used herein, “ameliorate,” “ameliorating,” “amelioration,” or “ameliorated” in reference to a disorder can refer to reducing the symptoms of the disorder, causing stable disease, or preventing progression of the disorder, For disorders such as cancer, this can include reducing the size of a tumor, reducing cancer cell growth or proliferation or cancer cell number, completely or partially removing the tumor (e.g., a complete or partial response), causing stable disease, preventing progression of the cancer (e.g., progression free survival), or any other effect on the cancer that would be considered by a physician to be a therapeutic.

As used herein, “administer,” administering,” or “administered” can refer to methods of introducing the compound, or pharmaceutically acceptable salt thereof, or modified cell composition, to a patient or subject, including, but not limited to, oral, intravenous, intramuscular, subcutaneous, or transdermal.

As used herein, “subject” or “patient,” can refer to any organism upon which the embodiments described herein may be used or administered, e.g., for experimental, diagnostic, prophylactic, and/or therapeutic purposes. Subjects or patients include, for example, animals. In some embodiments, the subject is mice, rats, rabbits, non-human primates, or humans. In some embodiments, the subject is a cow, sheep, pig, horse, dog, cat, primate or a human.

As used herein, “co-administration” can refer to the administration of more than one therapeutic agent in combination with another. Each reagent can be administered sequentially or concurrently, such that each reagent can be in the blood stream of an organism at the same time.

### Certain Polynucleotides

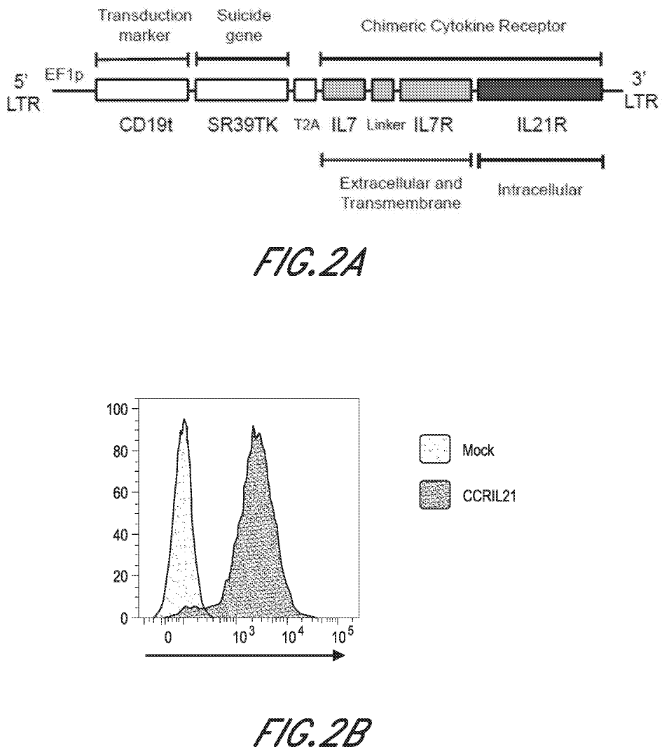

Some embodiments of the methods and compositions provided herein include a polynucleotide encoding a chimeric cytokine receptor polypeptide. In some embodiments, the chimeric cytokine receptor polypeptide can include a cytokine, such as IL-7, tethered to an extracellular domain and transmembrane domain of a type I cytokine receptor, such as an IL-7 receptor extracellular and transmembrane domains; and an intracellular domain of a type I cytokine receptor, such as an IL-21 receptor intracellular domain. In some such embodiments, the transmembrane domain links the extracellular receptor domain to the intracellular receptor domain. In some embodiments, the chimeric cytokine receptor polypeptide can include an IL-7 tethered to an extracellular IL-7 receptor domain; a transmembrane domain; and an intracellular IL-21 receptor domain, in which the transmembrane domain links the extracellular IL-7 receptor domain to the intracellular IL-21 receptor domain. An exemplary embodiment of such a chimeric cytokine receptor is depicted in FIG. 2A. In some embodiments, the IL-7 receptor domains are derived from IL-7 alpha chain domains.

In some embodiments, a polynucleotide can include a first nucleic acid encoding the IL-7. In some embodiments, a polynucleotide can include a second nucleic acid encoding a tether. In some embodiments, a polynucleotide can include a third nucleic acid encoding the extracellular IL-7 receptor domain, wherein the IL-7 is linked to the extracellular IL-7 receptor domain via the tether. In some embodiments, a polynucleotide can include a fourth nucleic acid encoding the IL-7 receptor transmembrane domain. In some embodiments, a polynucleotide can include a fifth nucleic acid encoding an intracellular IL-21 receptor domain.

In some embodiments, a polynucleotide can include a first nucleic acid encoding the IL-7. In some embodiments, the IL-7 is mammalian, such as human. In some embodiments, the first nucleic acid comprises a nucleotide sequence having a percentage identity with the nucleotide sequence of SEQ ID NO:02. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, a polynucleotide can include a second nucleic acid encoding a tether. In some embodiments, the tether can have a length greater than or equal to 2 amino acids and less than or equal to 50 amino acids, greater than or equal to 5 amino acids and less than or equal to 40 amino acids, greater than or equal to 10 amino acids and less than or equal to 35 amino acids, greater than or equal to 10 amino acids and less than or equal to 30 amino acids, or greater than or equal to 15 amino acids and less than or equal to 25 amino acids. In some embodiments, the second nucleic acid comprises a nucleotide sequence having a percentage identity with the nucleotide sequence of SEQ ID NO:03. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, a polynucleotide can include a third nucleic acid encoding the extracellular IL-7 receptor domain, wherein the IL-7 is linked to the extracellular IL-7 receptor domain via the tether. In some embodiments, the extracellular IL-7 receptor domain is mammalian, such as human. In some embodiments, the third nucleic acid comprises a nucleotide sequence having a percentage identity with the nucleotide sequence of SEQ ID NO:04. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, a polynucleotide can include a fourth nucleic acid encoding the transmembrane domain. In some embodiments, the transmembrane domain comprises an IL-7 receptor transmembrane domain, or an IL-21 receptor transmembrane domain. In some embodiments, the transmembrane domain comprises an IL-7 receptor transmembrane domain. In some embodiments, the IL-7 receptor transmembrane domain is mammalian, such as human. In some embodiments, the third and fourth nucleic acids together encode the extracellular IL-7 receptor domain and the IL-7 receptor transmembrane domain, and comprises a nucleotide sequence having a percentage identity with the nucleotide sequence of SEQ ID NO:04. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, a polynucleotide can include a fifth nucleic acid encoding an intracellular IL-21 receptor domain. In some embodiments, the intracellular IL-21 receptor domain is mammalian, such as human. In some embodiments, the fifth nucleic acid comprises a nucleotide sequence having a percentage identity with the nucleotide sequence of SEQ ID NO:05. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, a polynucleotide can also include an inducible promoter. In some such embodiment, transcription of a chimeric cytokine receptor can be induced in the presence of certain drugs, which interact with the inducible promoter.

In some embodiments, a polynucleotide can also encode an inducible cytotoxic gene. In some such embodiments, transcription of the cytotoxic gene can be induced to induce killing of the cell containing the polynucleotide. Examples of cytotoxic genes include genes encoding a protein such as a thymidine kinase, a thymidine kinase fused to thymidylate kinase, an oxidoreductase, a deoxycytidine kinase, a uracil phosphoribosyltransferase, a cytosine deaminase, or a cytosine deaminase fused to uracil phosphoribosyltransferase. In some embodiments, a thymidine kinase is preferred. In some such embodiments, the thymidine kinase comprises SR39TK. An example of a nucleotide sequence comprising SR39TK includes the nucleotide sequence of SEQ ID NO:07.

In some embodiments, a polynucleotide can include more than one protein encoding sequences. In some such embodiments, the polynucleotide can include a ribosomal skip sequence. An example of a ribosomal skip sequence includes a T2A skip sequence. An example of a nucleotide sequence comprising a T2A skip sequence includes the nucleotide sequence of SEQ ID NO:06.

In some embodiments, a polynucleotide can include a nucleic acid encoding a transduction marker. Such markers are useful to identify and obtain cells that have been successfully transduced with a polynucleotide, and successfully express the encoded marker. An example of a transduction marker includes a truncated CD19 (CD19t) molecule.

Some embodiments of the methods and compositions provided herein include a polypeptide encoded by a polynucleotide provided herein.

### Vectors

Some embodiments of the methods and compositions provided herein concern a vector comprising a polynucleotide described herein. In some embodiments, the vector is suitable for or configured for transduction into a cell. In some embodiments, the vector comprises a viral vector. Examples of viral vectors include a lentiviral vector, an adeno-associated viral vector, or an adenoviral vector. In some embodiments, the vector comprises a lentiviral vector.

In some embodiments, a vector can include a polynucleotide provided herein encoding a chimeric cytokine receptor, and a polynucleotide encoding a CAR.

### Cells

Some embodiments of the methods and compositions provided herein include a cell containing a polynucleotide provided herein, or a polypeptide encoded by a polynucleotide provided herein. In some embodiments, the cell also contains a polynucleotide encoding a CAR, or a CAR protein. In some embodiments, the cell is a T cell. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human.

### Therapeutic Methods

Some embodiments of the methods and compositions provided herein concern methods of treating or ameliorating a cancer in a subject. Some such methods include administering a cell provided herein to a subject in need thereof. In some embodiments, the cell includes chimeric cytokine receptor or a polynucleotide encoding the chimeric cytokine receptor, and a CAR or a polynucleotide encoding the CAR.

In some embodiments, the cancer can include a solid tumor such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the cancer comprises a brain cancer.

Some of the chimeric cytokine receptors provided herein can reduce or eliminate a need to supplement a therapy to a subject by co-administering a cytokine. For example, administration of a CAR T cell to a subject, which does not contain a chimeric cytokine receptor can include co-administration of an exogenous cytokine to further stimulate or activate the CAR T cells that have been administered to the subject. As described herein, T cells containing a chimeric cytokine receptor provided herein can be provided in a sufficiently stimulated and/or activated state in the absence of an exogenous cytokine, or a substantially reduced dose of an exogenous cytokine compared to a T cell not containing a chimeric cytokine receptor provided herein. In some embodiments, the treatment or amelioration of the cancer lacks co-administration of a cytokine to the subject. Some embodiments, can also include co-administering a cytokine to the subject, wherein the dose of the cytokine is reduced compared to the dose of the cytokine co-administered to a subject who has been administered a cell comprising a CAR, which lacks a chimeric cytokine receptor provided herein. In some such embodiments, the dose of a cytokine co-administered to a subject who has been administered a cell comprising a CAR and a chimeric cytokine receptor can be reduced in comparison to the dose of a cytokine co-administered to a subject who has been administered a cell comprising a CAR, which lacks a chimeric cytokine receptor by more than or more than about 10%, 20%, 30%, 40%, 50%, 60%, 70%, 80%, 90%, 100%, or an amount that is within a range defined by any two of the aforementioned percentages.

In some embodiments, administration of a CAR T cell containing a chimeric cytokine receptor provided herein improves the removal of tumor from a subject compared to the removal of a tumor in a subject administered a CAR T cell lacking a chimeric cytokine receptor provided herein.

In some such embodiments, administration of a CAR T cell containing a chimeric cytokine receptor provided herein reduces the volume of a of tumor in a subject compared to the volume of a tumor in a subject who has been administered a CAR T cell lacking a chimeric cytokine receptor provided herein. In some such embodiments, the volume of a of tumor in a subject administration of a CAR T cell containing a chimeric cytokine receptor provided herein compared to the volume of a tumor in a subject who has been administered a CAR T cell lacking a chimeric cytokine receptor provided herein can be reduced by more than or more than about 10%, 20%, 30%, 40%, 50%, 60%, 70%, 80%, 90%, 100%, or an amount that is within a range defined by any two of the aforementioned percentages.

In some such embodiments, administration of a CAR T cell containing a chimeric cytokine receptor provided herein reduces the volume of a of tumor in a subject at a greater rate that a reduction in the volume of a tumor in a subject who has been administered a CAR T cell lacking a chimeric cytokine receptor provided herein.

In some embodiments, administration of a CAR T cell containing a chimeric cytokine receptor provided herein increases overall survival of a subject compared to the overall survival of a subject administered a CAR T cell lacking a chimeric cytokine receptor provided herein.

In some of the foregoing embodiments, the cytokine is selected from IL-7, IL-15, or IL-21. In some embodiments, the cytokine comprises IL-21. In some embodiments, the cell is autologous to the subject. In some embodiments, the subject is mammalian, such as human.

### Methods of Preparing Populations of Cells

Some embodiments of the methods and compositions provided herein concern methods of preparing a population of cells comprising a CAR. Some such methods include (a) transducing a T cell with a polynucleotide encoding a chimeric cytokine receptor provided herein; (b) transducing the T cell with a polynucleotide encoding a CAR; and (c) culturing the transduced T cell under conditions sufficient to stimulate activation and expansion of the T cell, wherein the culture media comprises a reduced amount of an exogenous cytokine as compared to an amount sufficient to stimulate activation and expansion of the T cell lacking a chimeric cytokine receptor provided herein. In some embodiments, step (b) is performed before step (a). In some embodiments, step (a) and (b) are performed concurrently.

In some embodiments, the amount of an exogenous cytokine sufficient to stimulate activation and expansion of a T cell comprising a CAR and a chimeric cytokine receptor provided herein is reduced in comparison to the amount of an exogenous cytokine sufficient to stimulate activation and expansion of a T cell comparing a CAR and lacking a chimeric cytokine receptor provided herein by more than or more than about 10%, 20%, 30%, 40%, 50%, 60%, 70%, 80%, 90%, 100%, or by an amount that is within a range defined by any two of the aforementioned percentages. In some embodiments, the cytokine is selected from IL-7, IL-15, or IL-21. In some embodiments, the cytokine comprises IL-21. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian, such as human.

Some embodiments include a method of preparing a population of cells comprising a chimeric antigen receptor (CAR), comprising: (a) transducing a T cell with a polynucleotide encoding a chimeric cytokine receptor provided herein; (b) transducing the T cell with a polynucleotide encoding a CAR; and (c) culturing the transduced T cell under conditions to stimulate activation and expansion of the T cell. In some embodiments, T cells are grown using culture media which lacks an exogenous cytokine. In some embodiments, step (b) is performed before step (a). In some embodiments, step (a) and (b) are performed concurrently. In some embodiments, the cytokine is selected from IL-7, IL-15, or IL-21. In some embodiments, the cytokine comprises IL-21. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian, such as human.

### Kits and Systems

Some embodiments of the methods and compositions provided herein concern kits for preparing a population of T cells. In some embodiments, a kit can include a polynucleotide or vector provided herein.

Some embodiments of the methods and compositions provided herein include a system for preparing a population of T cells. In some embodiments, a system can include a polynucleotide or vector provided herein. In some embodiments, a system can also include a cell provided herein.

### EXAMPLES

### Example 1—Methods

Chimeric cytokine receptor for IL21 signaling (CCRIL21) construct design. The CCRIL21 construct included a single reading frame encoding a CD19 marker protein, followed by a T2A ribosomal skip sequence and a CCRIL21 protein (FIG. 2A).

Expression was driven by a human elongation factor 1α (EF1α) promoter. The CCRIL21 protein included an extracellular domain comprising an extracellular and transmembrane domain of an endogenous IL7R, which was tethered via a flexible linker sequence to the IL7 cytokine. On the intracellular side of the IL7R transmembrane domain was an intracellular domain of the endogenous IL21R. TABLE 1 lists nucleic elements of the chimeric cytokine receptor for IL21 signaling (CCRIL21). Each sequence in TABLE 1 was connected in the listed order to constitute the full CCRIL21 sequence. TABLE 2 lists additional nucleic acid elements useful with certain embodiments provided herein.

T cell production and culture. CD8+ T cells were isolated from human peripheral blood mononuclear cells (PBMCs) by magnetic activated cell sorting and subjected to a bead-based CD3/CD28 stimulation using Dynabeads (Life Technologies). Two days later, cells were transduced with lentivirus to introduce CCRIL21 and/or CAR transgenes. Between days 10 and 24 post-stimulation, methotrexate (MTX) was added to the CAR-transduced T cell cultures to select for CAR-expressing populations. Twenty-four days post-stimulation, CCRIL21-expressing cells were enriched by magnetic sorting for CD19 expression (FIG. 2B). Sorted populations were subjected to a rapid expansion protocol (REP). See e.g., Wang, X. et al. J. Immunother. 35, 689-701 (2012). MTX was again supplemented between days 5 and 14 of the REP. At 14 days post-REP, flow cytometry confirmed purified T cell populations expressing CCRIL21 and/or CAR constructs, and T cells were frozen down for later assays. T cells were thawed and put immediately into a second REP and assays were begun 14 days later.

Apoptotic status assay. Cells were stained with Annexin V and 7-AAD (BioLegend) to determine viability and apoptotic status. Cells were removed from culture and placed into 96-well round-bottom plates, washed twice with BioLegend Annexin V Staining Buffer, stained with Annexin V and 7-AAD, washed two more times with Annexin V Buffer and analyzed by flow cytometry within 12 hours.

Cell cycle assay. The Millipore Muse Cell Cycle Analysis Kit was used to quantify T cell mitotic activity. This kit was used for flow cytometric determination of the cell cycle stage of individual cells. Cells were gated into cell cycle stages based on DNA content via propidium iodide staining.

Cytotoxicity assay. Target cells were loaded with Chromium⁵¹ and co-cultured in variable ratios with T cell groups for 4 hours. Lysis of target cells was quantified by measurement of Chromium⁵¹ released into the supernatant (Cooper, L. J. N. et al. Blood 101, 1637-1644 (2003)).

Intracellular cytokine staining. T cells were co-cultured with EGFRvIII-expressing K562 cells (K562-vIII) at a 2:1 effector to target ratio for 6 hours. Two hours after co-culture start, a transport inhibitor cocktail was added to prevent T cell release of secretory proteins. At the end of co-culture, cells were stained for surface markers, fixed and permeabilized using the CytoFix/CytoPerm kit (BD) and finally intracellularly stained for secretory proteins.

Phospho-flow. Phosphorylation status of signaling proteins was assessed by flow cytometry as described in Krutzik, P. O. et al., Cytometry 55A, 61-70 (2003). Concentrated PFA was added directly to cell culture wells and cells were then permeabilized with 70% ethanol. Staining was performed using anti-phosphoSTAT antibodies (BioLegend).

Flow-based cytotoxicity assay with mixed targets. CAR T cells were co-incubated with a mixture of K562 cells expressing CD19t (off-target) and K562 cells expressing EGFRvIII (on-target). After co-incubation, populations were sorted via flow cytometry and killing of each target population was assessed using dye that selectively labels dead cells.

Mouse studies. In vivo anti-tumor efficacy of CCR-expressing CAR T cells was assessed using an orthotopic glioblastoma model according to IACUC-approved protocols. 10-12 week old male NSG mice were intracranially injected with 200,000 human U87 cells expressing GFP and firefly luciferase. Tumor engraftment was assessed using bioluminescent imaging for firefly luciferase luminescence upon sub-cutaneous injection with D-luciferin. Mice were subsequently sorted into experimental groups with 4-5 mice per group. Seven days later, 1×10⁶ T cells were intracranially injected directly into the site of tumor injection. Thereafter, mice were monitored for tumor progression via bioluminescent imaging until day 90.

### Example 2—CCRIL21 Recapitulated IL-21 Signaling Events

In CD8+ T cells, exogenous IL-21 can bind to an IL-21 receptor complexed with the common gamma-chain, and the heterodimeric receptor complex can induce IL-21 related signaling events including phosphorylation of STAT3 and STAT5.

Primary CD8+ human T cells were cultured without cytokines or with IL-21 (10 ng/mL) for 14 hours before analysis by flow cytometry for the presence of phospho-STAT3 and phospho-STAT5. As shown in FIG. 3, cells containing the CCRIL21 and not cultured with IL-21 demonstrated substantially similar levels of phosphorylated STAT3 and STAT5 as control cells not containing the CCRIL21 and cultured with IL-21. Thus, CCRIL21 recapitulated IL-21 signaling events in primary CD8+ human T cells.

### Example 3—CCRIL21 Mediated Cytokine-Independent Cell Cycle Progression and Survival

To assess the effects of CCRIL21 on T cell proliferation and survival in vitro, CCRIL21-expressing and Mock T cells were cultured in the absence of exogenous cytokines. Cell cycle progression was examined two days later (FIG. 4A), and apoptotic status of the cells was examined six days later (FIG. 4B). As a control, Mock T cells were also cultured with 10 ng/mL IL-21.

As shown in FIG. 4A, cells containing the CCRIL21 and not cultured with IL-21 had a substantially similar proportion of cells undergoing S phase and G2/M phase, as control cells not containing the CCRIL21 and cultured with IL-21. Moreover, the total percentage of cells containing the CCRIL21 and not cultured with IL-21 that were undergoing S phase and G2/M phase was significantly greater than the total percentage of control cells not cultured with IL-21 that were undergoing S phase and G2/M phase.

As shown in FIG. 4B, the total percentages of cells that were undergoing apoptosis or were dead were substantially similar for cells containing the CCRIL21 and not cultured with IL-21, and of control cells not containing the CCRIL21 and cultured with IL-21.

### Example 4—CCRIL21-Expressing CAR T Cells were Primed for Increased Cytotoxicity

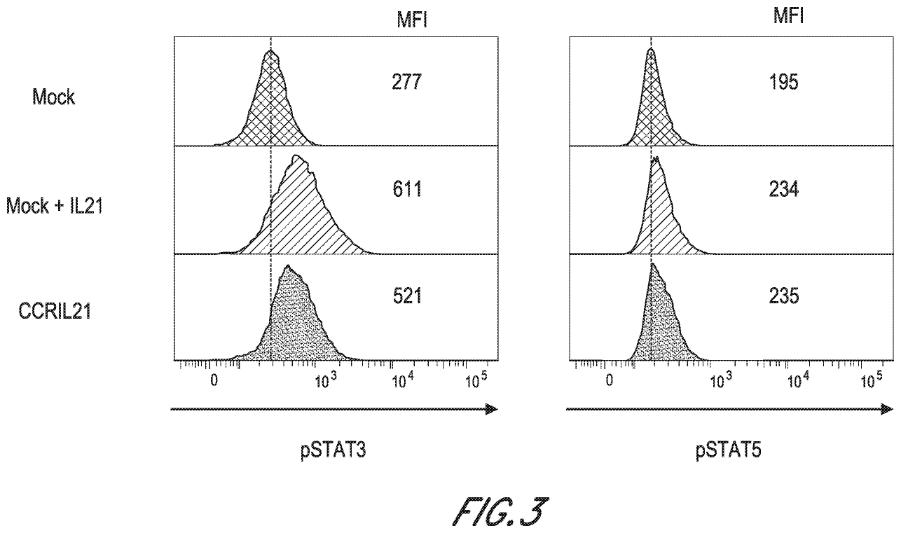

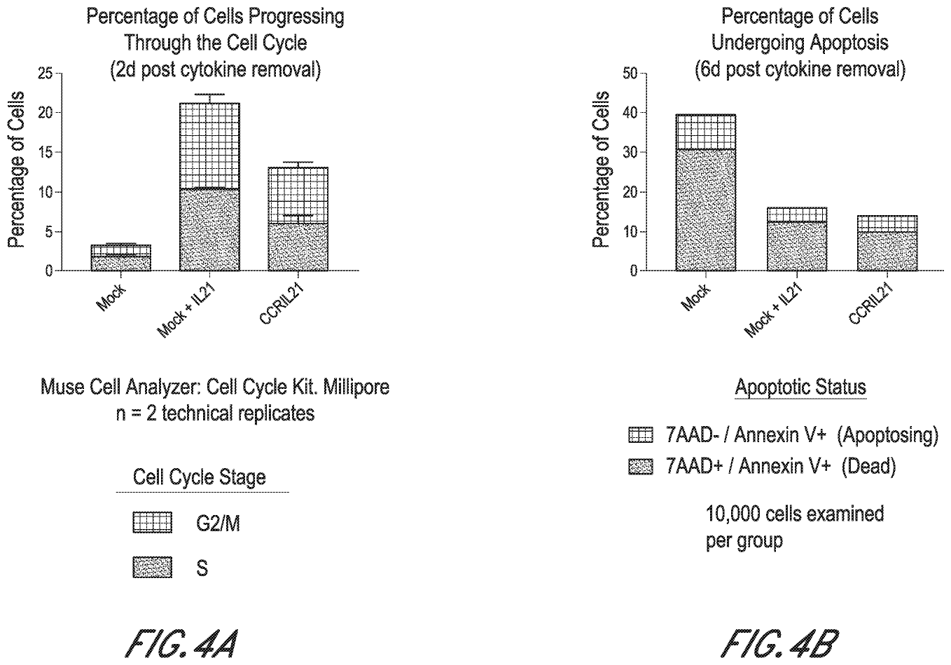

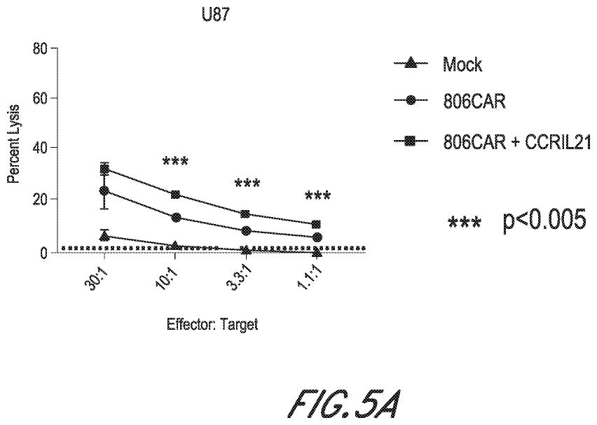

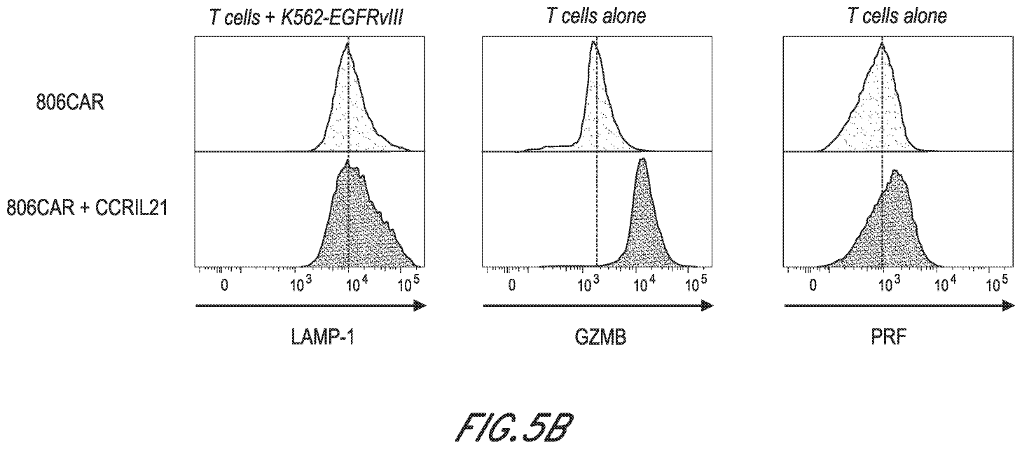

The activity of either T cells containing a CAR, which targets EGFRvIII (806CAR), or T cells containing the 806 CAR and CCRIL21 was determined in a cytotoxicity assay. CCRIL21-expressing CAR T cells show enhanced cytotoxicity in a 4-hour chromium release assay against the human glioblastoma cell line U87 (FIG. 5A).

Levels of proteins associated with cytotoxicity, including Lamp-1, Granzyme B and Perforin, were also determined. Surface presence of Lamp-1 was assessed after six hour co-incubation with K562-EGFRvIII cells and baseline expression of cytotoxic proteins Granzyme B and Perforin by flow cytometry (FIG. 5B). CCRIL21-expressing cells showed higher levels of a degranulation protein Lamp-1 (LAMP-1); and cytotoxic proteins, such as Granzyme B (GZMB) and Perforin (PRF).

### Example 5—CCRIL21-Primed Cytotoxicity is Restricted to CAR-Targeted Cells

Cytotoxic activity of either T cells containing the 806CAR, or T cells containing the 806 CAR and CCRIL21 was determined with target cells expressing either the target (EGFRvIII), or a non-target control CD19t. Cells were co-incubated with a mixture of K562 cells expressing CD19t (off-target) and K562 cells expressing EGFRvIII (on-target). After co-incubation, populations were sorted by flow cytometry, and killing of each target population was assessed using flow cytometry techniques.

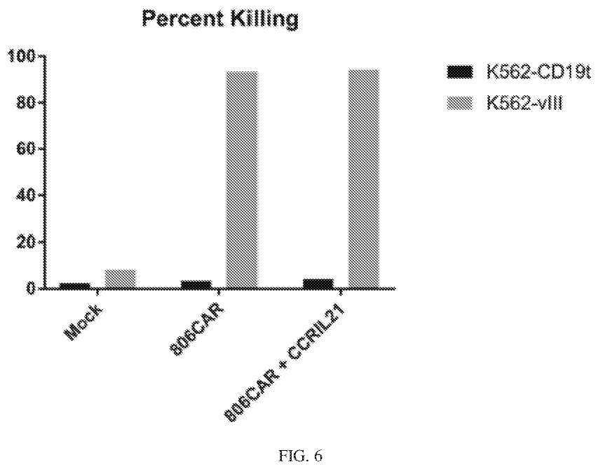

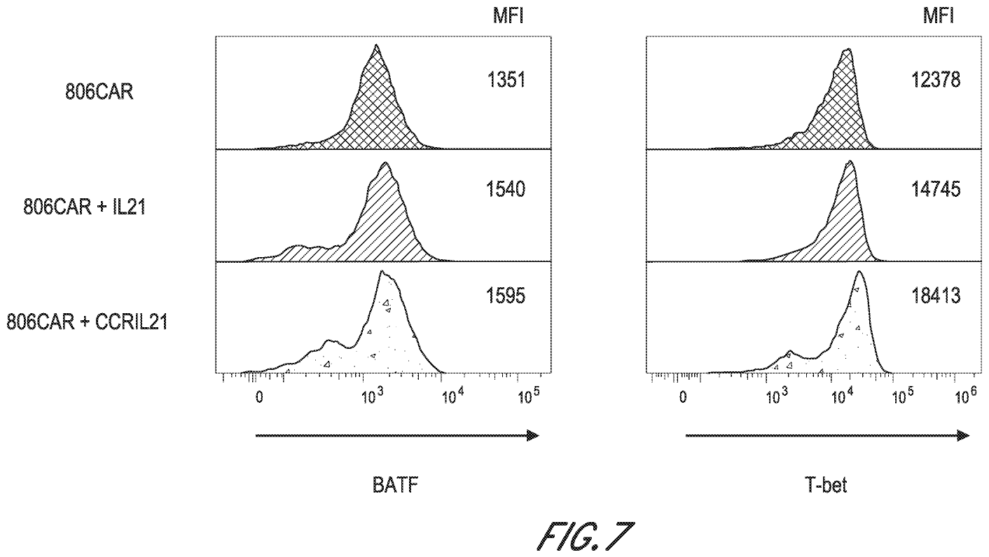

As shown FIG. 6, CCRIL21 did not lead to increased toxicity against off-target tumor cells, indicating that the increased cytotoxicity promoted by CCRIL21 was restricted to cells targeted by the CAR.

### Example 6—CCRIL21 Regulated Effector Function Through Key Transcription Factors in 806CAR T Cells

T cells containing either the 806CAR, or the 806 CAR and CCRIL21 were cultured with in a 1:1 ratio with K562 cells expressing EGFRvIII for 24 hours. Cultures containing T cells containing the 806CAR only were contacted with IL-21.

Levels of transcription factors, BATF and T-bet were determined (FIG. 7). BATF and T-bet are associated with the acquisition and maintenance of cytotoxic function. CCRIL21 recapitulated the effects of IL-21 on CAR T cells by upregulating transcription factors including BATF and T-bet. This demonstrated that CCRIL21-expressing CAR T cells were primed for increased cytotoxicity.

### Example 7—CCRIL21 Expression Increases Tumor Clearance by CAR T Cells In Vivo

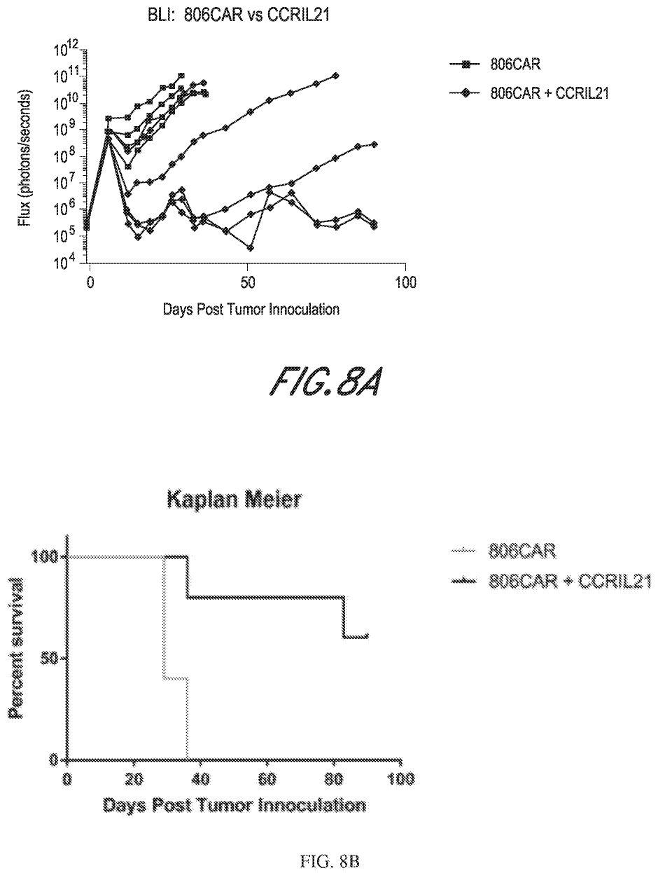

Mice were engrafted with intracranial human glioblastoma tumors. Seven days later, mice were intracranially administered T cells expressing a tumor-specific CAR (806CAR). 806CAR T cells expressing CCRIL21 led to greater tumor removal, as measured by bioluminescent imaging (FIG. 8A). Furthermore, mice treated with 806CAR T cells expressing CCRIL21 survived significantly longer than mice treated with T cells expressing the CAR alone (FIG. 8B).

TABLE 1

| Feature | |
|---|---|
| [SEQ ID NO] | Sequence |
| GMCSF signal | ATGCTTCTCCTGGTGACAAGCCTTCTGCTCTGTGAGTTACC |
| sequence | ACACCCAGCATTCCTCCTGATCCCA |
| [SEQ ID NO: 01] | |
| human IL7 | GATTGTGATATTGAAGGTAAAGATGGCAAACAATATGAG |
| cytokine sequence | AGTGTTCTAATGGTCAGCATCGATCAATTATTGGACAGCA |
| (start codon | TGAAAGAAATTGGTAGCAATTGCCTGAATAATGAATTTAA |
| removed) | CTTTTTTAAAAGACATATCTGTGATGCTAATAAGGAAGGT |
| [SEQ ID NO: 02] | ATGTTTTTATTCCGTGCTGCTCGCAAGTTGAGGCAATTTCT |
| | TAAAATGAATAGCACTGGTGATTTTGATCTCCACTTATTAA |
| | AAGTTTCAGAAGGCACAACAATACTGTTGAACTGCACTGG |
| | CCAGGTTAAAGGAAGAAAACCAGCTGCCCTGGGTGAAGC |
| | CCAACCAACAAAGAGTTTGGAAGAAAATAAATCTTTAAA |
| | GGAACAGAAAAAACTGAATGACTTGTGTTTCCTAAAGAGA |
| | CTATTACAAGAGATAAAAACTTGTTGGAATAAAATTTTGA |
| | TGGGCACTAAAGAACAC |
| Flexible Linker | GGAGGCGGTGGGAGCGGAGGCGGTGGGAGC |
| sequence | |
| (Glycine₄Serine₁)₂ | |
| [SEQ ID NO: 03] | |
| human IL7R | ATGACAATTCTAGGTACAACTTTTGGCATGGTTTTTTCTTT |
| extracellular | ACTTCAAGTCGTTTCTGGAGAAAGTGGCTATGCTCAAAAT |
| domain and | GGAGACTTGGAAGATGCAGAACTGGATGACTACTCATTCT |
| transmembrane | CATGCTATAGCCAGTTGGAAGTGAATGGATCGCAGCACTC |
| domain | ACTGACCTGTGCTTTTGAGGACCCAGATGTCAACATCACC |
| [SEQ ID NO: 04] | AATCTGGAATTTGAAATATGTGGGGCCCTCGTGGAGGTAA |
| | AGTGCCTGAATTTCAGGAAACTACAAGAGATATATTTCAT |
| | CGAGACAAAGAAATTCTTACTGATTGGAAAGAGCAATATA |
| | TGTGTGAAGGTTGGAGAAAAGAGTCTAACCTGCAAAAAA |
| | ATAGACCTAACCACTATAGTTAAACCTGAGGCTCCTTTTG |
| | ACCTGAGTGTCATCTATCGGGAAGGAGCCAATGACTTTGT |
| | GGTGACATTTAATACATCACACTTGCAAAAGAAGTATGTA |
| | AAAGTTTTAATGCACGATGTAGCTTACCGCCAGGAAAAGG |
| | ATGAAAACAAATGGACGCATGTGAATTTATCCAGCACAAA |
| | GCTGACACTCCTGCAGAGAAAGCTCCAACCGGCAGCAATG |
| | TATGAGATTAAAGTTCGATCCATCCCTGATCACTATTTTAA |
| | AGGCTTCTGGAGTGAATGGAGTCCAAGTTATTACTTCAGA |
| | ACTCCAGAGATCAATAATAGCTCAGGGGAGATGGATCCTA |
| | TCTTACTAACCATCAGCATTTTGAGTTTTTTCTCTGTCGCT |
| | CTGTTGGTCATCTTGGCCTGTGTGTTATGG |
| human IL21R | AGCCTGAAAACACACCCTCTGTGGCGGCTGTGGAAGAAA |
| intracellular | ATCTGGGCCGTGCCATCTCCTGAGCGGTTCTTCATGCCTCT |
| domain | GTACAAGGGCTGCAGCGGCGACTTCAAGAAATGGGTCGG |
| [SEQ ID NO: 05] | AGCCCCTTTTACCGGCAGCTCTCTGGAACTTGGACCTTGG |
| | AGCCCTGAGGTGCCCAGCACACTGGAAGTGTACAGCTGTC |
| | ACCCTCCTAGAAGCCCCGCCAAGAGACTGCAGCTGACAGA |
| | GCTGCAAGAGCCTGCCGAGCTGGTGGAATCTGATGGCGTG |
| | CCCAAGCCTAGCTTCTGGCCCACAGCTCAGAATAGCGGCG |
| | GCTCTGCCTACAGCGAGGAAAGAGATAGACCCTACGGCCT |
| | GGTGTCCATCGACACCGTGACAGTGCTGGATGCCGAGGGA |
| | CCTTGTACCTGGCCTTGTAGCTGCGAGGACGATGGCTACC |
| | CTGCTCTGGATCTGGACGCTGGCCTTGAGCCTTCTCCAGG |
| | ACTGGAAGATCCTCTGCTGGACGCCGGAACAACCGTGCTG |
| | TCTTGTGGCTGTGTGTCTGCCGGATCTCCTGGACTTGGAGG |
| | CCCTCTGGGAAGCCTGCTGGATAGACTGAAACCTCCTCTG |
| | GCCGACGGCGAAGATTGGGCTGGTGGACTTCCTTGGGGCG |
| | GAAGATCTCCAGGCGGAGTGTCTGAGTCTGAAGCCGGTTC |
| | TCCACTGGCCGGCCTGGACATGGATACCTTCGATTCTGGC |
| | TTCGTGGGCAGCGACTGTAGCAGCCCTGTGGAATGCGACT |
| | TCACAAGCCCTGGCGACGAGGGCCCACCTAGAAGCTATCT |
| | GAGACAGTGGGTCGTGATCCCTCCACCTCTGTCTAGTCCT |
| | GGACCTCAGGCTTCT |

TABLE 2

| Feature | |
|---|---|
| [SEQ ID | |
| NO] | Sequence |
| T2A | GGCGGCGGAGAGGGCAGAGGAAGTCTTCTAACATGCGGT |
| [SEQ ID | GACGTGGAGGAGAATCCCGGCCCT |
| NO: 06] | |
| SR39TK | ATGCCCACGCTACTGCGGGTTTATATAGACGGTCCCCACG |
| gene | GGATGGGGAAAACCACCACCACGCAACTGCTGGTGGCCCT |
| [SEQ ID | GGGTTCGCGCGACGATATCGTCTACGTACCCGAGCCGATG |
| NO: 07] | ACTTACTGGCGGGTGCTGGGGGCTTCCGAGACAATCGCGA |
| | ACATCTACACCACACAACACCGCCTCGACCAGGGTGAGAT |
| | ATCGGCCGGGGACGCGGCGGTGGTAATGACAAGCGCCCA |
| | GATAACAATGGGCATGCCTTATGCCGTGACCGACGCCGTT |
| | CTGGCTCCTCATATCGGGGGGGAGGCTGGGAGCTCACATG |
| | CCCCGCCCCCGGCCCTCACCATCTTCCTCGACCGCCATCCC |
| | ATCGCCTTCATGCTGTGCTACCCGGCCGCGCGGTACCTTAT |
| | GGGCAGCATGACCCCCCAGGCCGTGCTGGCGTTCGTGGCC |
| | CTCATCCCGCCGACCTTGCCCGGCACCAACATCGTGCTTG |
| | GGGCCCTTCCGGAGGACAGACACATCGACCGCCTGGCCAA |
| | ACGCCAGCGCCCCGGCGAGCGGCTGGACCTGGCTATGCTG |
| | GCTGCGATTCGCCGCGTTTACGGGCTACTTGCCAATACGG |
| | TGCGGTATCTGCAGTGCGGCGGGTCGTGGCGGGAGGACTG |
| | GGGACAGCTTTCGGGGACGGCCGTGCCGCCCCAGGGTGCC |
| | GAGCCCCAGAGCAACGCGGGCCCACGACCCCATATCGGG |
| | GACACGTTATTTACCCTGTTTCGGGCCCCCGAGTTGCTGGC |
| | CCCCAACGGCGACCTGTATAACGTGTTTGCCTGGGCCTTG |
| | GACGTCTTGGCCAAACGCCTCCGTTCCATGCACGTCTTTAT |
| | CCTGGATTACGACCAATCGCCCGCCGGCTGCCGGGACGCC |
| | CTGCTGCAACTTACCTCCGGGATGGTCCAGACCCACGTCA |
| | CCACCCCCGGCTCCATACCGACGATATGCGACCTGGCGCG |
| | CACGTTTGCCCGGGAGATGGGGGAGGCTAAC |

### Example 8—CCRIL21-Expressing B7H3CAR T Cells are Primed for Increased Cytotoxicity Against Tumor Cells

In order to assess whether, CCRIL21-expressing CAR T cells are primed for increased cytotoxicity against tumor cells, CD8+B7H3CAR T cells were co-cultured with K562 tumor cells at a ratio of 2 tumor cells to each T cell. An Incucyte S3 live cell fluorescent imager was used to monitor tumor cell growth over the course of a week.

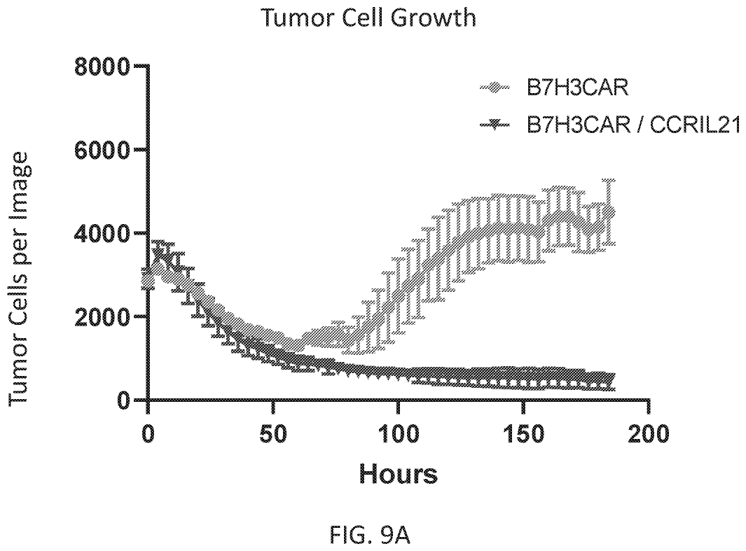

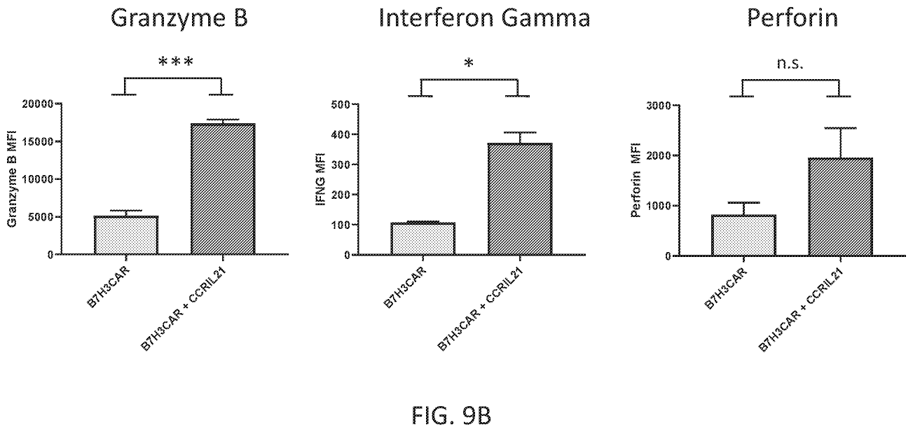

As shown by the data in FIG. 9A, B7H3CAR T cells were eventually overrun by tumor cells, but CCRIL21-expressing CAR T cells were able to suppress tumor growth over the course of study.

These data demonstrate the sustained cytotoxic capacity of CCRIL21-expressing CAR T cells. Furthermore, the results show that CCRIL21 augments cytotoxicity in B7H3CAR T cells as well as in 806CAR T cells.

### Example 9—CCRIL21-Expressing B7H3CAR T Cells are Primed for Increased Cytotoxicity Due to Increased Expression of Effector Proteins

In order to assess whether CCRIL21-Expressing CAR T Cells are primed for increased cytotoxicity due to increased cytotoxic capacity, CD8+B7H3CAR T cells were co-cultured with K562 tumor cells before intracellular flow cytometric analysis of effector protein expression.

The graphs in FIG. 9B display the average of median fluorescent intensity (MFI) across three biological replicates. Error bars reflect standard deviation, and a one-way ANOVA statistical analysis was performed (n.s.=not significant, *p<0.05, ***p<0.005).

As shown by the data, CCRIL21-expressing CAR T cells exhibited higher levels of cytotoxic proteins Granzyme B, Interferon gamma, and Perforin as compared to B7H3CAR T cells.

These findings demonstrate that CCRIL21 primes cytotoxic capacity by supporting higher expression levels of effector proteins in B7H3CAR T cells, as found in 806CAR T cells.

### Example 10—CCRIL21 Regulates Effector Function Through Key Transcription Factors in B7H3CAR T Cells

In order to determine whether CCRIL21 regulates effector function through key transcription factors, expression of transcription factors Tbet and BATF were assessed using intracellular flow cytometry.

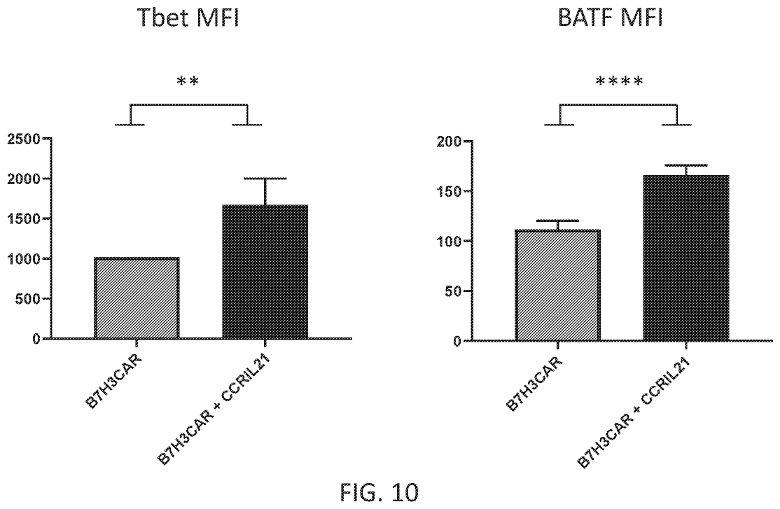

The graphs in FIG. 10 display the average of median fluorescent intensity (MFI) across three biological replicates. Error bars reflect standard deviation, and a one-way ANOVA statistical analysis was performed (**p<0.01, ****p<0.001). CCRIL21-expressing CAR T cells exhibit statistically higher levels of Tbet and BATF.

The data suggest that, as in 806CAR T cells, CCRIL21 affects global changes in gene expression in B7H3CAR T cells by regulating the expression of key transcription factors.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A polynucleotide encoding a chimeric cytokine receptor polypeptide, wherein the chimeric cytokine receptor polypeptide comprises:
an IL-7 tethered to an extracellular IL-7 receptor domain;
a transmembrane domain; and
an intracellular IL-21 receptor domain, wherein the transmembrane domain links the extracellular IL-7 receptor domain to the intracellular IL-21 receptor domain.

2-59. (canceled)


SEQUENCE LISTING (7 sequences)

SEQ ID NO: 1 - DNA, 66 bp, Artificial Sequence
  Synthetic; GMCSF signal sequence
atgcttctcctggtgacaagccttctgctctgtgagttaccacacccagcattcctcctgatccca

SEQ ID NO: 2 - DNA, 456 bp, Artificial Sequence
  Synthetic; human IL7 cytokine sequence (start codon removed)
gattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 3 - DNA, 30 bp, Artificial Sequence
  Synthetic; Flexible Linker sequence (Glycine4Serine1)2
ggaggcggtgggagcggaggcggtgggagc

SEQ ID NO: 4 - DNA, 792 bp, Artificial Sequence
  Synthetic; human IL7R extracellular domain and transmembrane domain
atgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcatctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatgg

SEQ ID NO: 5 - DNA, 855 bp, Artificial Sequence
  Synthetic; human IL21R intracellular domain
agcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagagcctgccgagctggtggaatctgatggcgtgcccaagcctagcttctggcccacagctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcttct

SEQ ID NO: 6 - DNA, 63 bp, Artificial Sequence
  Synthetic; T2A
ggcggcggagagggcagaggaagtcttctaacatgcggtgacgtggaggagaatcccggccct

SEQ ID NO: 7 - DNA, 993 bp, Artificial Sequence
  Synthetic; SR39TK gene
atgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccatcttcctcgaccgccatcccatcgccttcatgctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaac